(19) United States
(12) Patent Application Publication
(10) Pub. No.: US 2024/0131027 A1
(43) Pub. Date: Apr. 25, 2024

(54) PHARMACEUTICAL COMPOSITION COMPRISING NOVEL SD911 COMPOUND AS ACTIVE INGREDIENT FOR PREVENTION OR TREATMENT OF TRANSPLANT REJECTION

(71) Applicant: THE CATHOLIC UNIVERSITY OF KOREA INDUSTRY-ACADEMIC COOPERATION FOUNDATION, Seoul (KR)

(72) Inventors: Chul Woo YANG, Seoul (KR); Dongyun SHIN, Seoul (KR); Sun Woo LIM, Seoul (KR); Mi La CHO, Seoul (KR); Yoo Jin SHIN, Seoul (KR); Seon Yeong LEE, Seoul (KR); A Ram LEE, Jinju-si (KR)

(73) Assignee: THE CATHOLIC UNIVERSITY OF KOREA INDUSTRY-ACADEMIC COOPERATION FOUNDATION, Seoul (KR)

(21) Appl. No.: 18/286,677

(22) PCT Filed: Apr. 6, 2022
(86) PCT No.: PCT/KR2022/004950

§ 371 Date: Oct. 12, 2023

(30) Foreign Application Priority Data
Apr. 12, 2021 (KR) 10-2021-0047049

(51) Int. Cl. A61K 31/502; A61P 37/06

(52) U.S. Cl. CPC A61K 31/502; A61P 37/06

(57) ABSTRACT

The present invention relates to a pharmaceutical composition comprising a novel compound as an active ingredient for prevention or treatment of transplant rejection. The novel compound SD911 of the present invention was identified to repress the migration of immune cells, increase the engraftment and viability of a grafted tissue, and regulate immune cells. In addition, it was found in a kidney transplantation model that the compound restrained human PBMC from suppressing the engraftment and damaging renal tissues and downregulated the expression of the inflammatory cytokine IL-17, with the consequent effective prevention and treatment of transplant rejection.

### TECHNICAL FIELD

The present invention relates to a pharmaceutical composition for prevention or treatment of transplantation rejection comprising a novel SD911 compound as an active ingredient.

### BACKGROUND ART

An immune system needs to differentiate between self and non-self. When the immune system does not differentiate between self and non-self, the immune system destroys cells and tissues of the body, and as a result, causes autoimmune diseases. Regulatory T cells actively suppress the activation of the immune system to prevent pathological self-reactivity and consequently to prevent autoimmune diseases. The regulatory T cells (Treg) are a class of CD4+CD25+ T cells that suppress the activity of other immune cells. The immune system is well-equipped with a device capable quickly identifying and rapidly destroying foreign species, pathogens, or inflamed tissue. The immune system has been a major barrier to tissue, organ and cell transplantation as well as gene therapy. The major problems were generally chronic immunosuppression, encapsulation and immune isolation. Unwanted side effects of chronic immunosuppression include increased susceptibility to opportunistic infections and tumor formation.

The mechanisms by which T cell responses to foreign (allogeneic or xenogeneic) proteins or cells or organs operate are significantly well known. Antigen presenting cells (APCs) are introduced into an inflammation or injury site (triggered by transplantation), and a peripheral T cell repertoire constantly monitors for the presence (allogeneic or xenogeneic) of foreign tissue or pathogens. Once these warning signals are recognized, APCs ingest the protein, digest the protein, and are presented to a host immune system. Allogeneic or syngeneic tumor cells have been engineered to express viral IL-10, which induced a local immune response against the tumor, but this treatment did not affect the rejection of non-transformed tumors at a distance (Suzuki et al., 1995). Topically administered IL-10 is thought to alter the T cell repertoire reactive to transplanted cells into a Th2 phenotype that is not cytolytic, but may even be protective.

Organ, tissue or cell transplantation may be used to save the lives of patients suffering from various types of diseases. Allotransplantation of human organs such as kidney, liver, heart, kidney, lung, and pancreas, tissues such as skin, bone marrow, and the like has been already commonly performed in hospitals as a method of treating intractable diseases such as late-stage organ failure. In addition, xenotransplantation using a non-human mammal as a donor is also being actively researched as a method to replace the shortage of allotransplant donors. In particular, recently, transplantation of stem cells capable of regenerating themselves permanently and differentiating into various types of cells constituting the body under appropriate conditions has emerged as one of cell replacement treatments for various intractable diseases. According to the National Organ Transplantation Center, about 20,000 patients a year are waiting for organ transplantation in Korea, but the number of donations is reported to be less than 10% of the demand. Also, in the case of the United States, one person requiring organs are added to a waiting list every 16 minutes, but 11 people on the waiting list die every day without being able to undergo surgery. In addition to this situation, the development of life science technology serves as the background for the development of xenotransplantation technology. In 2010, a total of 28,664 transplants were performed in the United States, including 16,899 kidney transplants, 6291 liver transplants, 2333 heart transplants, and 1770 lung transplants (Engels et. al., 2011, JAMA, 306(17):1891-1901). Significant improvements have been made in immunosuppressive therapy and pre- and post-transplantation patient care, but graft rejection still affects approximately 60% of transplanted individuals. Thus, the graft rejection is a risk factor for loss of a plurality of grafts, with graft rejection observed in 40% or less of transplanted individuals within the first year after transplantation (Jain et al., 2000, Ann Surg. 232(4):490-500). Acute rejection is also a known risk factor for progressing to chronic rejection, and accordingly, detection and treatment of acute rejection episodes as fast as possible are a major goal to minimize graft damage and arrest downstream rejection episodes. In most cases, an adaptive immune response to the transplanted tissue is a major obstacle to successful transplantation. The rejection is caused by an immune response to alloantigens on the graft, and the alloantigen is a protein that varies from person to person within the species and accordingly, recognized as foreign by the recipient (Janeway, et al., 2001, Immunobiology: The Immune System in Health and Disease. 5th edition. New York: Garland Science).

Over the past decade, advances in surgical techniques, immunosuppressive therapies, and infectious monitoring and treatment have revolutionized the survival of patients and grafts. However, despite these successes, graft recipients still exhibit much higher morbidity and mortality than the general population. Although this is partially due to the effects of chronic allograft damage, the main cause is comorbidity affected by chronic immunosuppressive drug use (Soulillou and Giral 2007, Transplantation 72 (Suppl 12): S89-93, Hourmant et al., 1998, Lancet 351: 623-628; Halloran, 2004, N Engl J Med 351: 2715-2729).

Immunosuppression-related toxicities may be in a significant level. For example, a number of studies in adult liver transplant recipients have demonstrated a time-dependent serial decline in a kidney function according to exposure to immunosuppressive therapy. Other important complications of long-term immunosuppression include new-onset diabetes after transplantation (NODAT), hypertension, hyperlipidemia and the need for statin therapy (Srinivas et al., 2008, CJASN: (Supplement 2) S101-S116]). To rectify this situation, research priorities in organ transplantation are shifting from the search for new potent immunosuppressive drugs to strategies to minimize immunosuppression, and it is a goal to allow transplanted tissue to be maintained for a long time without continued immunosuppression.

Meanwhile, currently used immunosuppressants are divided into corticosteroid, antimetabolite, calcineurin inhibitor, mammalian target of rapamycin inhibitor, antibody, etc., but these immunosuppressants exhibit an immunosuppressive effect by blocking the proliferation or activation of T cells of the immune system at different stages (Dalai, P. et al. Int. J. Nephrol. Renovasc. Dis. 3:107-115 (2010)). T cells, the main target of immunosuppressive drugs, are generated in the human thymus and mainly differentiate into type 1 helper cells (Th1) involved in cell-mediated immunity or type 2 helper cells (Th2) involved in humoral immunity. It has been known that the two T cell populations keep each other in check so as not to be overactivated, and when the balance is broken, abnormal reactions such as autoimmunity or hypersensitivity reactions occur.

In addition, new types of T cells such as immunoregulatory T cells (Treg) and Th17 capable regulating immune responses have been known. Treg may regulate Th1 cell activity, suppress the functions of abnormally activated immune cells, and regulate inflammatory responses. On the other hand, Th17 cells secrete IL-17, and maximize the signal of the inflammatory response to accelerate the progression of the disease. Recently, these Treg or Th17 have greatly emerged as new targets for immune disease treatment, and various immunomodulatory therapeutic agents are being studied (Wood, K. J. et al., Nat. Rev. Immunol. 12 (6):417-430, 2012; Miossec, P. et al., Nat. Rev. Drug Discov. 11 (10):763-776, 2012; Noack, M. et al., Autoimmun. Rev. 13 (6):668-677, 2014).

Since existing immunosuppressants that non-specifically suppress T cells are generally accompanied by side effects, such as cytotoxicity, infection due to immunosuppression, diabetes, tremor, headache, diarrhea, hypertension, nausea, and renal dysfunction, there is a disadvantage that a long-term treatment effect is difficult to sustain. In order to reduce serious side effects and increase the effect of immunosuppressive treatment, methods of combining or replacing immunosuppressive agents with different mechanisms of action have been attempted, especially in the field of organ transplantation, but treatment using an optimized single compound is still incomplete.

Therefore, there is an urgent need to develop new immunosuppressive or immunomodulatory therapies capable of reducing the side effects of existing immunosuppressants and improving the therapeutic effect, and to discover new immunosuppressant candidates that are safer and have fewer side effects.

### DISCLOSURE

### Technical Problem

An object of the present invention is to provide a pharmaceutical composition for prevention or treatment of transplantation rejection, including a novel compound or a pharmaceutically acceptable salt thereof as an active ingredient.

Another object of the present invention is to provide a composition for immunosuppression after transplantation including the compound or a pharmaceutically acceptable salt thereof as an active ingredient.

Yet another object of the present invention is to provide a health functional food for prevention or improvement of transplantation rejection, including the compound or a pharmaceutically acceptable salt thereof as an active ingredient.

Yet another object of the present invention is to provide a method for treatment of transplantation rejection, including administering the compound or a pharmaceutically acceptable salt thereof to a subject.

### Technical Solution

In order to achieve the object, an object of the present invention provides a pharmaceutical composition for prevention or treatment of transplantation rejection, including a novel compound or a pharmaceutically acceptable salt thereof as an active ingredient.

In addition, another aspect of the present invention provides a composition for immunosuppression after transplantation including the compound or a pharmaceutically acceptable salt thereof as an active ingredient.

In addition, yet another aspect of the present invention provides a health functional food for prevention or improvement of transplantation rejection, including a novel compound or a pharmaceutically acceptable salt thereof as an active ingredient.

In addition, yet another aspect of the present invention provides a method for treatment of transplantation rejection, including administering the compound or a pharmaceutically acceptable salt thereof to a subject.

### Advantageous Effects

According to the present invention, the novel compound SD911 was identified to suppress the migration of immune cells, increase the engraftment and viability of a grafted tissue, and regulate immune cells. In addition, it was found in a kidney transplantation model that the compound had a transplantation rejection effect by suppressing the engraftment of human PBMC, damage to renal tissues, and the expression of the inflammatory cytokine IL-17, and thus, the compound may be usefully used in related industries.

### DESCRIPTION OF DRAWINGS

FIG. 1A is a diagram confirming the suppression of cell migration by treating immune cells derived from the spleen of a mouse with SD911.

FIG. 1B is a diagram quantifying the suppression of cell migration by treating immune cells derived from the spleen of a mouse with SD911.

FIG. 2A is a schematic diagram of an observation method after transplantation to confirm the viability of a graft according to SD911 treatment in a skin transplantation animal model.

FIG. 2B is a diagram confirming the viability of a graft according to SD911 treatment in a skin transplantation animal model.

FIG. 2C is a diagram quantifying the viability of a graft according to SD911 treatment in a skin transplantation animal model.

FIG. 3A is a diagram confirming and quantifying changes in INFγ positive cells according to SD911 treatment in a skin transplantation animal model by flow cytometry.

FIG. 3B is a diagram confirming and quantifying changes in IL-4 positive cells according to SD911 treatment in a skin transplantation animal model by flow cytometry.

FIG. 3C is a diagram confirming and quantifying changes in IL-17 positive cells according to SD911 treatment in a skin transplantation animal model by flow cytometry.

FIG. 3D is a diagram confirming and quantifying changes in CD4+CD25+Foxp3 positive cells according to SD911 treatment in a skin transplantation animal model by flow cytometry.

FIG. 4 is a diagram confirming the suppression of HAL according to SD911 treatment in kidney organoids.

FIG. 5A is a diagram confirming the suppression of PODXL and ABC-positive cell expression according to SD911 treatment in kidney organoids by flow cytometry.

FIG. 5B is a diagram confirming the suppression of LTL and ABC-positive cell expression according to SD911 treatment in kidney organoids by flow cytometry.

FIG. 5C is a diagram confirming the suppression of ECAD and ABC-positive cell expression according to SD911 treatment in kidney organoids by flow cytometry.

FIG. 5D is a diagram confirming the suppression of PODXL and DR-positive cell expression according to SD911 treatment in kidney organoids by flow cytometry.

FIG. 5E is a diagram confirming the suppression of LTL and DR-positive cell expression according to SD911 treatment in kidney organoids by flow cytometry.

FIG. 5F is a diagram confirming the suppression of ECAD and DR-positive cell expression according to SD911 treatment in kidney organoids by flow cytometry.

FIG. 6 is a diagram schematically illustrating a process of constructing a kidney transplantation avatar model and treating a drug.

FIG. 7A is a diagram confirming the construction of a kidney transplantation avatar model according to injection of normal human PBMC and kidney transplantation rejection patient PBMC by human PBMC engraftment.

FIG. 7B is a diagram confirming the construction of a kidney transplantation avatar model according to injection of normal human PBMC and kidney transplantation rejection patient PBMC at a serum creatinine concentration.

FIG. 8A is a diagram confirming damage to renal tissue in a kidney transplantation avatar model according to injection of normal human PBMC and kidney transplantation rejection patient PBMC by H&E staining.

FIG. 8B is a diagram quantifying damage to renal tissue in a kidney transplantation avatar model according to injection of normal human PBMC and kidney transplantation rejection patient PBMC.

FIG. 9A is a diagram confirming infiltration of CD4 positive cells in a kidney transplantation avatar model according to injection of normal human PBMC and kidney transplantation rejection patient PBMC by immunohistochemical staining.

FIG. 9B is a diagram quantifying infiltration of CD4 positive cells in a kidney transplantation avatar model according to injection of normal human PBMC and kidney transplantation rejection patient PBMC.

FIG. 10A is a diagram confirming infiltration of IL-17 positive cells in a kidney transplantation avatar model according to injection of normal human PBMC and kidney transplantation rejection patient PBMC by immunohistochemical staining.

FIG. 10B is a diagram quantifying infiltration of IL-17 positive cells in a kidney transplantation avatar model according to injection of normal human PBMC and kidney transplantation rejection patient PBMC.

### BEST MODE FOR THE INVENTION

The present invention provides a pharmaceutical composition for prevention or treatment of transplantation rejection, including a compound represented by Chemical Formula 1 below or a pharmaceutically acceptable salt thereof as an active ingredient.

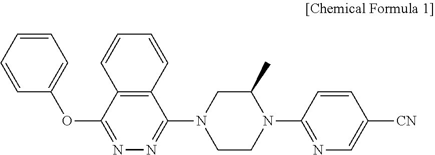 [SMILES: C[C@@H]1CN(c2nnc(Oc3ccccc3)c3ccccc23)CCN1c1ccc(C#N)cn1]

The compound may be synthesized through a process as shown in the following Reaction Scheme, but is not limited thereto:

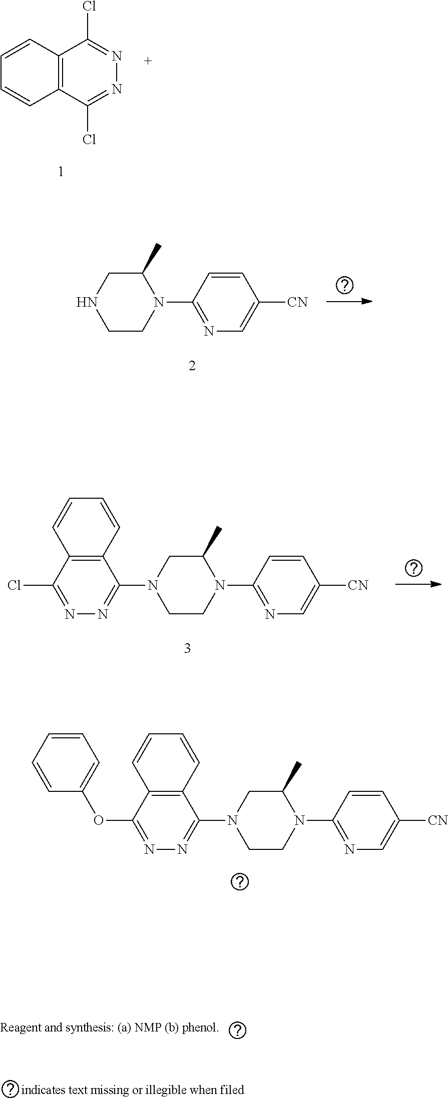 [SMILES: Clc1nnc(Cl)c2ccccc12 ; C[C@@H]1CNCCN1c1ccc(C#N)cn1 ; C[C@@H]1CN(c2nnc(Cl)c3ccccc23)CCN1c1ccc(C#N)cn1 ; C[C@@H]1CN(c2nnc(Oc3ccccc3)c3ccccc23)CCN1c1ccc(C#N)cn1]

The pharmaceutically acceptable salt may include an acid addition salt formed by a pharmaceutically acceptable free acid, and the free acid may use organic acids and inorganic acids. The organic acids may include citric acid, acetic acid, lactic acid, tartaric acid, maleic acid, fumaric acid, formic acid, propionic acid, oxalic acid, trifluoroacetic acid, benzoic acid, gluconic acid, metasulfonic acid, glycolic acid, succinic acid, 4-toluenesulfonic acid, glutamic acid, and aspartic acid, but are not limited thereto. In addition, the inorganic acids may include hydrochloric acid, hydrobromic acid, sulfuric acid, phosphoric acid, and the like, but are not limited thereto.

As used herein, the term “prevention” refers to any action that suppresses the symptoms of a specific disease or delays its progression by administering the composition of the present invention.

As used herein, the term “treatment” refers to any action that improves or beneficially changes the symptoms of a specific disease by administering the composition of the present invention.

As used herein, the term “transplantation rejection” occurs when a tissue to be transplanted is rejected by the immune system of a transplant recipient, and refers to a symptom in which the tissue to be transplanted is destroyed or inflammation is induced.

As used herein, the term “organ transplantation rejection” refers to a reaction in which the immune system of the recipient recognizes the transplanted tissue after transplantation as non-self and attacks and removes the transplanted organ. The most important factor involved in transplantation rejection is a major histocompatibility complex (MHC), and it is known that a minor histocompatibility complex is also involved in the transplantation rejection. The rejection involves both a cell-mediated immune response and a humoral immune response. The cell-mediated reaction starts when the lymphocytes of the recipient meet a donor MHC of the transplanted organ (CD4 T cell-type II MHC molecule, or CD8 T cell-type I MHC molecule). The activated T cells secrete cytokines, increase vascular permeability, and cause the infiltration of monocytes such as macrophages. As a result, microvascular damage, tissue ischemia, and destruction of grafted tissues and cells occur.

The composition according to the present invention may include a pharmaceutically effective dose of compound or extract alone or include one or more pharmaceutically acceptable carriers, excipients or diluents. The pharmaceutically effective dose refers to an amount sufficient to prevent, improve, and treat symptoms of the disease.

In addition, the “pharmaceutically acceptable” refers to a composition that is physiologically acceptable and does not cause an allergic reaction, such as gastrointestinal disorder, dizziness, etc., or a similar reaction thereto when administered to humans.

The pharmaceutical composition of the present invention may further include an adjuvant in addition to the active ingredient. The adjuvant may be used with any adjuvant known in the art without limitation, but further include, for example, Freund's complete adjuvant or incomplete adjuvant to increase the effect thereof.

The pharmaceutical composition according to the present invention may be prepared in the form of incorporating the active ingredient into a pharmaceutically acceptable carrier. Here, the pharmaceutically acceptable carrier includes carriers, excipients and diluents commonly used in a pharmaceutical field. The pharmaceutically acceptable carrier that may be used in the pharmaceutical composition of the present invention is not limited thereto, but may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil.

The pharmaceutical composition of the present invention may be formulated and used in the form of oral formulations, such as powders, granules, tablets, capsules, suspensions, emulsions, syrups, aerosols, etc., external preparations, suppositories, and sterile injectable solutions according to a conventional method.

The formulations may be prepared by using diluents or excipients, such as a filler, an extender, a binder, a wetting agent, a disintegrating agent, a surfactant, etc., which are generally used. Solid formulations for oral administration include tablets, pills, powders, granules, capsules, etc., and these solid formulations may be prepared by mixing at least one excipient, for example, starch, calcium carbonate, sucrose, lactose, gelatin, and the like with the active ingredient. Further, lubricants such as magnesium stearate and talc may be used in addition to simple excipients. Liquid formulations for oral administration may correspond to suspensions, oral liquids, emulsions, syrups, and the like, and may include various excipients, for example, a wetting agent, a sweetener, an aromatic agent, a preserving agent, and the like, in addition to water and liquid paraffin which are commonly used diluents. Formulations for parenteral administration include sterilized aqueous solutions, non-aqueous solvents, suspensions, emulsions, lyophilized agents, and suppositories. As the non-aqueous solution and the suspension, propylene glycol, polyethylene glycol, vegetable oil such as olive oil, injectable ester such as ethyl oleate, and the like may be used. As a base of the suppository, witepsol, Tween 61, cacao butter, laurinum, glycerogelatin, and the like may be used.

The pharmaceutical composition of the present invention may be administered to a subject through various routes. All methods of administration may be expected, and the pharmaceutical composition may be administered, for example, oral, intravenous, intramuscular, subcutaneous, and intraperitoneal injection.

The dose of the pharmaceutical composition according to the present invention is selected in consideration of the age, body weight, sex, and physical condition of the subject. It is obvious that the concentration of the active ingredient included in the pharmaceutical composition may be variously selected according to a subject, and preferably included in the pharmaceutical composition at a concentration of 0.01 μg/ml to 5,000 μg/ml. When the concentration is less than 0.01 μg/ml, pharmacological activity may not be shown, and when the concentration exceeds 5,000 μg/ml, toxicity to the human body may be exhibited.

According to an example of the present invention, the compound or the pharmaceutically acceptable salt thereof may be administered at a concentration of 10 to 300 μM.

According to an example of the present invention, the transplantation rejection may be at least one kind of transplantation rejection selected from the group consisting of cells, blood, tissues and organs, preferably organ transplantation rejection. The organ transplantation rejection may be at least one kind selected from the group consisting of bone marrow transplant, heart transplant, corneal transplant, intestinal transplant, liver transplant, lung transplant, pancreas transplant, kidney transplant, and skin transplantation rejections, preferably kidney transplant, but is not limited thereto.

According to an example of the present invention, the transplantation rejection may include graft-versus-host disease (GVHD).

The “graft-versus-host disease (GVHD)” of the present invention is rejection after transplantation mediated with T cells. In general, since regulator T cells (Treg) are important for regulating the graft-versus-host disease and the immune response regulation of Treg plays an important role in immune tolerance, it is known that Treg play an important role in the treatment of the graft-versus-host disease. Specifically, it is known that it is important for the treatment of graft-versus-host disease to suppress the differentiation or activity of allogeneic reactive T cells by increasing the differentiation or activity of Treg (Clinical Immunology (2009) 133, 22-26).

According to an example of the present invention, the composition may suppress the migration of immune cells.

According to an example of the present invention, the composition may increases the viability of a graft.

According to an example of the present invention, the composition may regulate immune cells, and the immune cells are T cells, but are not limited thereto.

According to an example of the present invention, the T cells may be one or more T cells selected from the group consisting of Th1, Th2, Th17 and Treg, but are not limited thereto.

According to an example of the present invention, the regulation of the immune cells may be to suppress the differentiation or activity of one or more T cells selected from the group consisting of Th1, Th2 and Th17, or to increase the differentiation or activity of Treg.

According to an example of the present invention, the composition may suppress the expression of HLA induced by cytokines.

According to an example of the present invention, the composition may suppress tissue damage, and the tissue may include at least one selected from the group consisting of bone marrow, heart, cornea, intestine, liver, lung, pancreas, kidney, and skin, preferably a kidney, but is not limited thereto.

According to an example of the present invention, the composition may suppress the infiltration of immune cells.

In addition, the present invention provides a composition for immunosuppression after transplantation including a SD911 compound as an active ingredient.

Further, the present invention provides a health functional food for prevention or improvement of transplantation rejection, including a SD911 compound as an active ingredient.

As used herein, the “improvement” means all actions that at least reduce parameters associated with conditions to be treated, for example, the degree of symptoms.

In addition to containing the active ingredient of the present invention, the food composition of the present invention may contain various flavoring agents or natural carbohydrates as additional ingredients like conventional food compositions.

Examples of the above-mentioned natural carbohydrates may include general sugars, such as monosaccharides, for example, glucose, fructose and the like; disaccharides, for example, maltose, sucrose and the like; and polysaccharides, for example, dextrin, cyclodextrin and the like, and sugar alcohols such as xylitol, sorbitol, erythritol, and the like. The above-mentioned flavoring agents may be advantageously used with natural flavoring agents (thaumatin), stevia extract (e.g., rebaudioside A, glycyrrhizin, etc.), and synthetic flavoring agents (saccharin, aspartame, etc.). The food composition of the present invention may be formulated in the same manner as the pharmaceutical composition to be used as a functional food or added to various foods. The food that may add the composition of the present invention may include, for example, beverages, meat, chocolate, foods, confectionery, pizza, ramen, other noodles, gums, candies, ice creams, alcohol beverages, vitamin complexes, health food supplements, and the like.

In addition, the food composition may contain various nutrients, vitamins, minerals (electrolytes), flavoring agents such as synthetic and natural flavoring agents, coloring agents and enhancers (cheese, chocolate, etc.), pectic acid and salts thereof, alginic acid and salts thereof, organic acid, a protective colloidal thickener, a pH adjusting agent, a stabilizer, a preservative, glycerin, alcohol, a carbonic acid agent used in a carbonated drink, and the like, in addition to the extract as the active ingredient. In addition, the food composition of the present invention may contain pulp for preparing natural fruit juice, fruit juice beverages, and vegetable beverages.

The functional food composition of the present invention may be prepared and processed in the form of tablets, capsules, powders, granules, liquids, pills, etc. for the purpose of prevention or treatment of transplantation rejection. In the present invention, the “health functional food composition” refers to food produced and processed using raw materials or ingredients with functionality, which are useful for the human body according to the Art on Health Functional Foods No. 6727, and means food taken for adjusting nutrients for the structures and functions of the human body or obtaining a useful effect on health applications such as physiological actions. The health functional food of the present invention may include conventional food additives, and the suitability as the food additive is determined by the specifications and standards for the corresponding item in accordance with the general rules of the Food Additive Codex, general test methods, and the like approved by the Food and Drug Administration, unless otherwise specified. The items disclosed in the “Food Additives Codex” may include, for example, chemical composites such as ketones, glycine, calcium citrate, nicotinic acid, cinnamic acid, and the like; natural additives such as desensitizing dye, licorice extract, crystal cellulose, Kaoliang color, guar gum, and the like; mixed formulations such as sodium L-glutamic acid formulations, noodle additive alkali agents, preservative formulations, tar color formulations, etc. For example, the health functional food in the form of tablets may formed by granulating a mixture obtained by mixing the active ingredient of the present invention with an excipient, a binder, a disintegrant, and other additives, and then compression-molding the mixture by adding a slip modifier and the like, or directly compressing the mixture. In addition, the health functional food in the form of tablets may also contain a flavors enhancer or the like as needed. Among health functional foods in the form of capsules, hard capsules may be prepared by filling a mixture mixed with the active ingredient of the present invention and additives such as excipients into conventional hard capsules, and soft capsules may be prepared by filling a mixture mixed with the active ingredient of the present invention and additives such as excipients into capsule bases such as gelatin. The soft capsules may contain a plasticizer such as glycerin or sorbitol, a colorant, a preservative, and the like if necessary. The health functional food in the form of pills may be prepared by molding a mixture mixed with the active ingredient of the present invention and an excipient, a binder, a disintegrant, and the like by existing known methods, and may also be coated with white sugar or other coating agents or surface-coated with materials such as starch and talc, if necessary. The health functional food in the form of granules may be produced by granulating a mixture of mixing the active ingredient of the present invention with an excipient, a binder, a disintegrant, and the like by existing known methods and may contain a flavoring agent, a flavors enhancer, and the like if necessary.

In addition, the present invention provides a method for treatment of transplantation rejection, including administering the compound or a pharmaceutically acceptable salt thereof to a subject.

The treatment method of the present invention includes administering to a subject a therapeutically effective dose of the compound or the pharmaceutically acceptable salt thereof. It is preferred that a specific therapeutically effective dose for a specific subject is differently applied depending on various factors including the kind and degree of a response to be achieved, a specific composition including whether other agents are used in some cases, the age, body weight, general health conditions, sex, and diet of a subject, an administration time, an administration route, a secretion rate of the composition, a duration of treatment, and a drug used in combination or simultaneously with the specific composition, and similar factors well known in the medical field. A daily dose may be 0.0001 to 100 mg/kg, preferably 0.01 to 100 mg/kg, based on the amount of the pharmaceutical composition of the present invention, and may be administered 1 to 6 times a day. However, it is obvious to those skilled in the art that the dosage or dose of each active ingredient does not cause side effects by including the content of each active ingredient too high. Therefore, the effective dose of the composition suitable for the purpose of the present invention is preferably determined in consideration of the aforementioned matters.

The subject is applicable to any mammal, and the mammal includes not only humans and primates, but also livestock such as cattle, pig, sheep, horse, dog, and cat.

The compound of the present invention or the pharmaceutically acceptable salt thereof may be administered to mammals such as mice, rats, livestock, and humans through various routes. All methods of administration may be expected, and for example, the pharmaceutical composition may be administered by oral, rectal or intravenous, intramuscular, subcutaneous, intrauterine dural or intracerebroventricular injection.

### MODES FOR THE INVENTION

### <Preparation Example 1> Preparation of Compound SD911

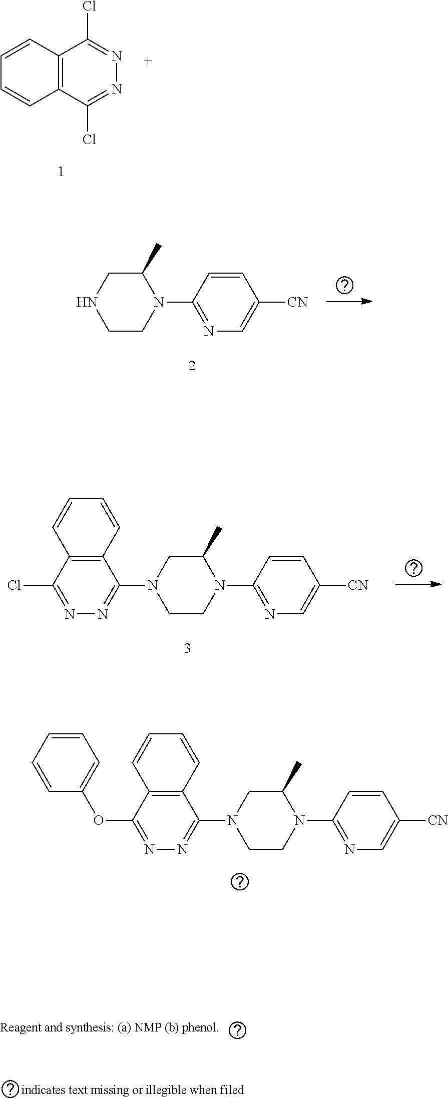 [SMILES: Clc1nnc(Cl)c2ccccc12 ; C[C@@H]1CNCCN1c1ccc(C#N)cn1 ; C[C@@H]1CN(c2nnc(Cl)c3ccccc23)CCN1c1ccc(C#N)cn1 ; C[C@@H]1CN(c2nnc(Oc3ccccc3)c3ccccc23)CCN1c1ccc(C#N)cn1]

6-((R)-4(1-chlorophthalazin-4-yl)-2-methylpiperazin-1-yl)pyridine-3-carbonitrile (3)

Compound (2) (502 mg, 2.48 mmol) was added with 1,4-diclonaphthalein (494 mg, 2.48 mmol) in a NMP (3.5 ml) solution. After 24 hours, the reaction mixture was diluted with ethyl acetate, combined with an organic phase, washed with water, and concentrated under reduced pressure. The residue was purified by flash column chromatography (ethyl acetate:N-hexane=1:2) on silica gel to obtain Compound (3) (49.4 mg, yield=5.46%). MS (ESI) [M+H]+365

6-((R)-2-methyl-4-(1-phenoxyphthalazin-4-yl)piperazin-1-yl)pyridine-3-carbonitrile (SD-911)

Compound (3) (15.7 mg, 0.043 mmol) and phenol (5.3 mg, 0.059 mmol) were added with K₂CO₃ (16 mg, 0.1161 mmol) in a DMF solvent (0.05 ml). After 24 hours, the reaction mixture was diluted with ethyl acetate, combined with an organic phase, washed with water, and concentrated under reduced pressure. The residue was purified by flash column chromatography (ethyl acetate:N-hexane=1:2) on silica gel to obtain a compound called “SD-911” (14.5 mg, yield=80%).

1H NMR (600 MHz, CDCl3) 6 (ppm)=8.43 (d, J=6.0 Hz, 1H), 8.40 (t, J=6.0 Hz, 1H), 8.15 (m, 1H), 7.92 (m, 2H), 7.64 (m, 1H), 7.42 (m, 2H), 7.28 (d, J=12 Hz, 2H), 7.23 (m, 1H), 6.64 (d, J=6.0 Hz, 1H), 4.74 (bs, 1H), 4.32 (d, J=18 Hz, 1H), 3.80 (d, J=18 Hz, 1H), 3.67 (d, J=18 Hz, 1H), 3.54 (m, 1H), 3.29 (m, 1H), 3.20 (d, J=6.0 Hz, 1H), 3.15 (m, 1H), 1.52 (d, J=6.0Hz, 3H); HMS (ESI) [M+H]+423.

### <Example 1> Confirmation of Suppression of Migration of Immune Cells by SD911

The present inventors screened a novel compound SD911 by using activity inhibition analysis of inflammatory cytokines in immune cells of a sphingosin 1-phosphate lyase (SPL) inhibitor, and tried to confirm an immune cell migration inhibitory effect of the screened SD911. Specifically, single cells (immune cells) were isolated from the spleens of normal C57/BL6 mice, and the isolated immune cells were cultured in 4 μM trans wells (upper compartment). For migration of the cultured immune cells, stromal cell-derived factor 1 (SDF1) was treated at a concentration of 20 ng/ml to stimulate the lower compartment. The immune cells moved to the lower compartment by stromal cell-derived factor 1 (SDF1), and at this time, whether the migration was inhibited by SD911 was analyzed.

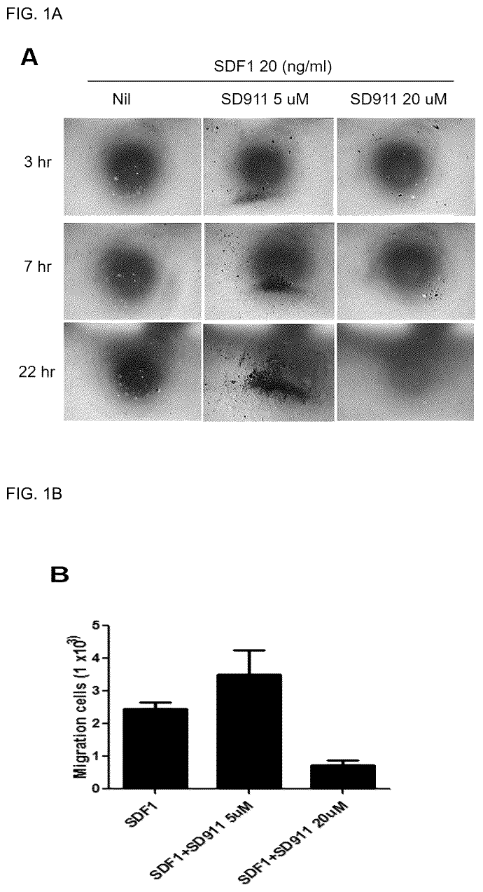

As a result, it was confirmed that when SD911 was treated at a concentration of 20 μM, the migration of immune cells induced by SDF1 was blocked, and it was confirmed that SD911 inhibited the migration of immune cells to target cells (tissues) (FIGS. 1A and 1B).

### <Example 2> Confirmation of SD911 Effect in Allogeneic Skin Transplantation Model

### <2-1> Production of Skin Transplantation Animal Model

In order to confirm A therapeutic effect of SD911 of the present invention on transplantation rejection, an allogenic skin transplantation animal model was used. Specifically, as the allogenic skin transplantation animal model, the tail skins of male donor C57BL/c mice (Orient Bio Inc.) were exfoliated and transplanted onto the back skins of male recipient BALB/c mice (Orient Bio Inc.). One day before transplant, the recipient mice were divided into two groups, a control group (Saline group) administered intraperitoneally with physiological saline, and an experimental group (SD911 group) administered with SD911 at a concentration of 200 μg/mice.

### <2-2> Confirmation of Skin Transplant Viability

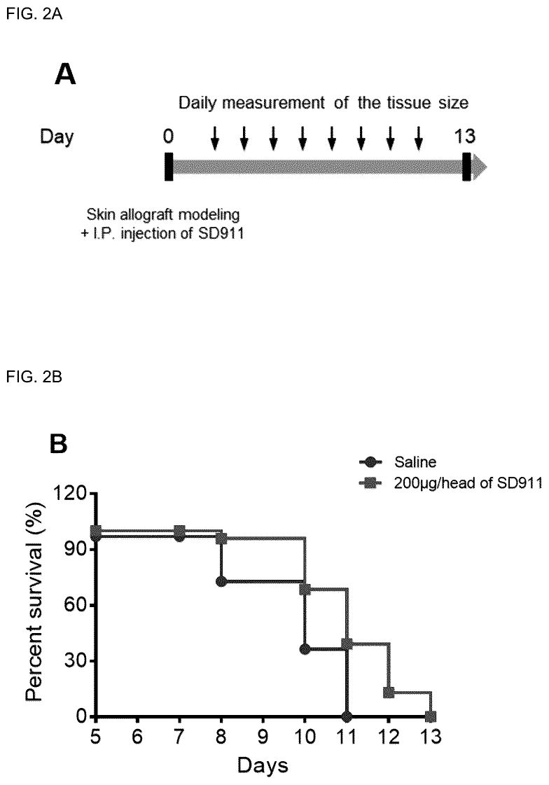

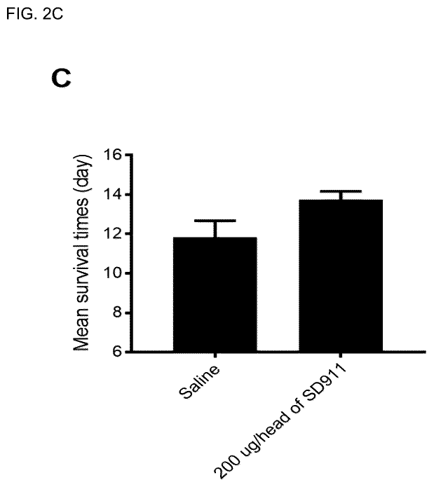

The skin changes of the control group or the experimental group (SD911 group) treated with SD911 at a concentration of 200 μg/mice were observed daily, and the viability was calculated by calculating the rejection when scab formation or skin atrophy of 70% or more occurred on the transplanted skin. A drug administration schedule was illustrated in FIG. 2A.

As a result, as illustrated in FIG. 2B, compared to the transplanted skin of the control group, it was confirmed that the viability of the transplanted skin of the mouse treated with SD911 at a concentration of 200 μg/mice significantly increased, and it was confirmed that the survival time was also significantly increased (FIG. 2C).

### <2-3> T Cell Subtype Analysis

Some of the recipient mice that had undergone the skin transplant were humanely sacrificed after 5 days, the spleens were extracted, and immune cells were isolated from the spleens. In order to analyze T cell subtypes in the isolated immune cells, flow cytometry was performed, and to identify pro-inflammatory T cells, the number of CD4+, IFN+, IL4+ and IL17+ positive cells was analyzed. In addition, regulatory T cells (Treg) were identified as CD4+CD25+Foxp3+ positive cells.

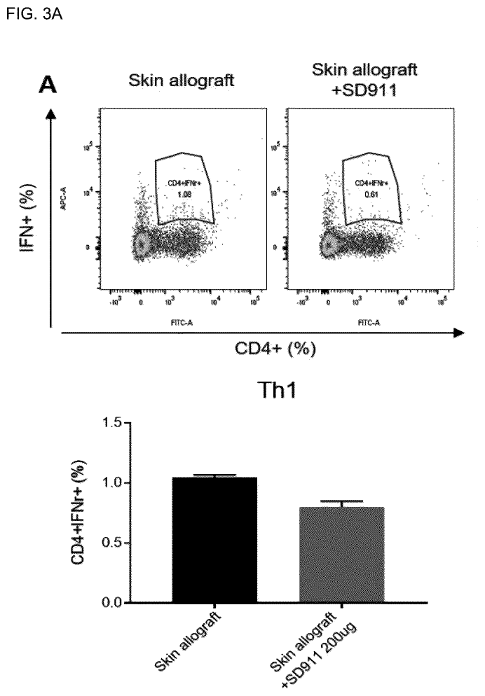

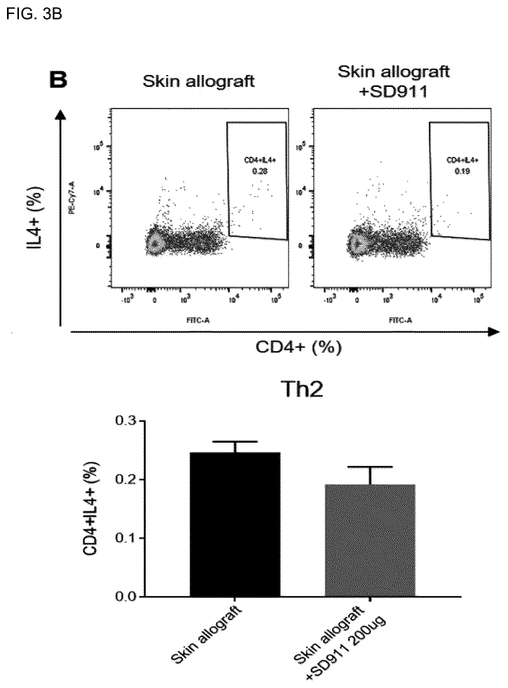

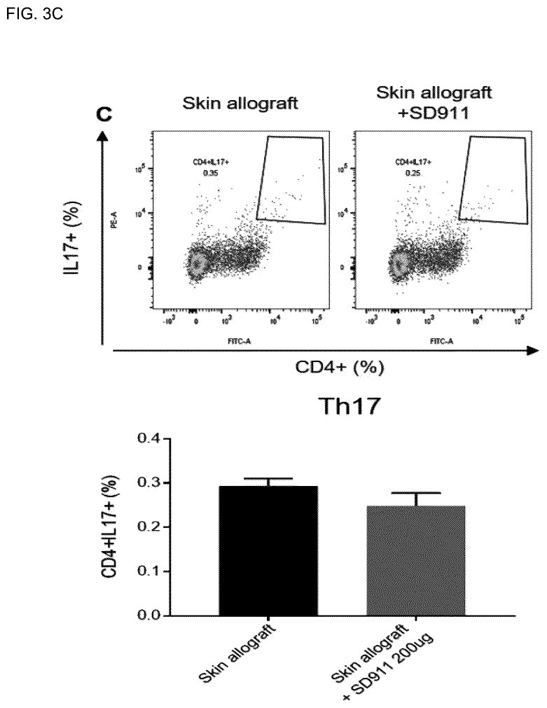

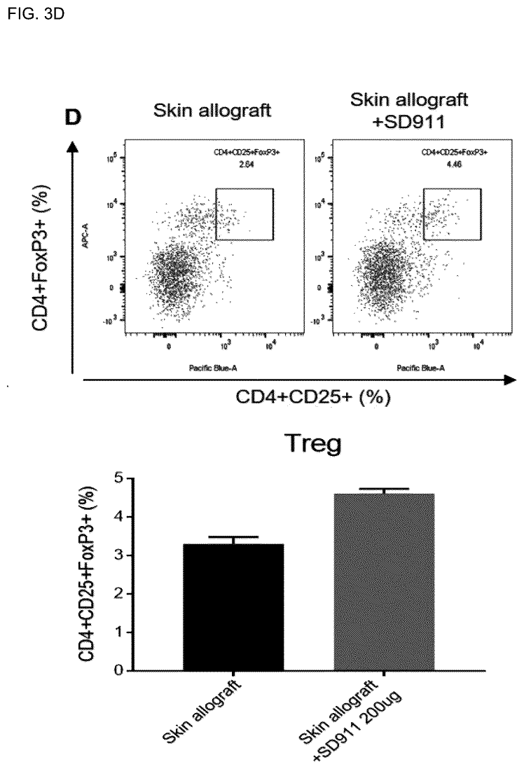

As a result, as illustrated in FIG. 3, compared to the control group, it was confirmed that when treated with SD911 at a concentration of 200 μg/mice, the number of Th1, Th2, and Th17 cells associated with inflammation significantly decreased (FIGS. 3A to 3C), and Treg increased (FIG. 3D).

### <Example 3> Confirmation of SD911 Effect in Kidney Organoids

### <3-1> Confirmation of HLA Inhibition in Kidney Organoids

WTC-11 cells, standard normal human induced pluripotent stem cells (iPSC) were differentiated into kidney organoids and treated with IFNγ, a cytokine known to induce transplantation rejection to construct an in vitro rejection model increasing human leukocyte antigen (HLA). Thereafter, the constructed kidney organoids were treated with 50 μM of SD911.

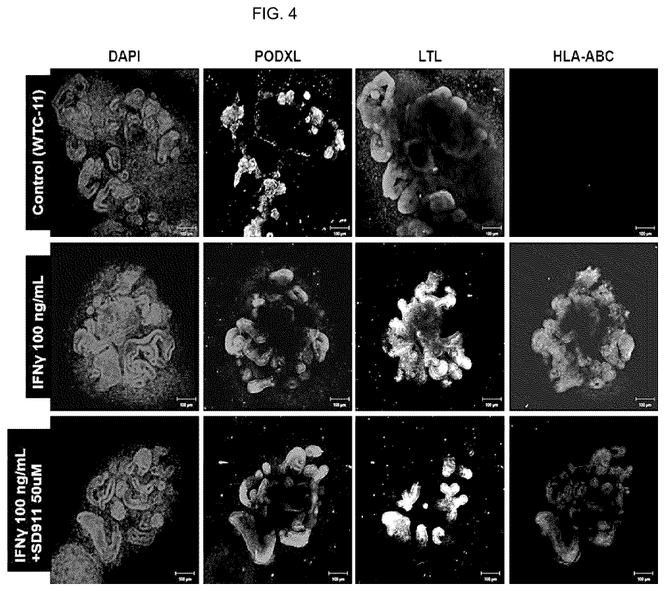

As a result, as illustrated in FIG. 4, it was confirmed that when the kidney organoids were treated with IFNγ, the HLA was increased, and when treated with SD911, the increased HAL was suppressed.

### <3-2> Flow Cytometry in Kidney Organoids

In the kidney organoids prepared in Example 3-1, when treated with the cytokines TNF-α and INFγ and when treated with SD911 at a concentration of 50 μg/mL, changes in cell patterns were analyzed by flow cytometry. Specifically, the patterns of nephron markers PODXL, LTL, ECAD and double positive cells in kidney organoids were analyzed.

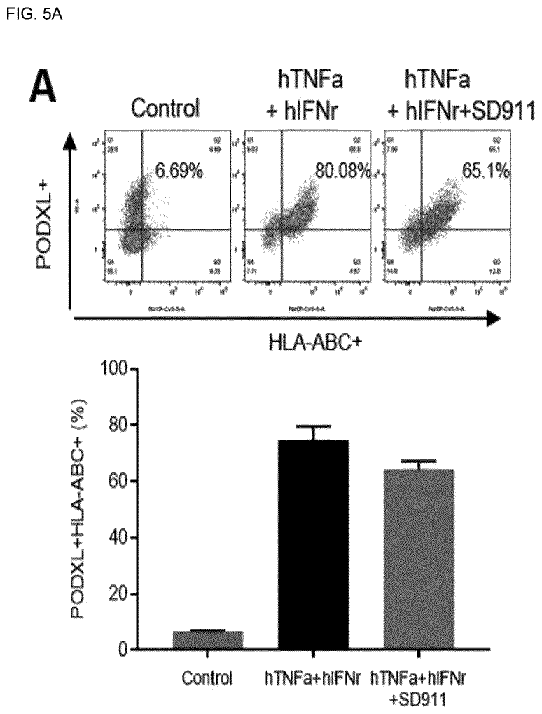

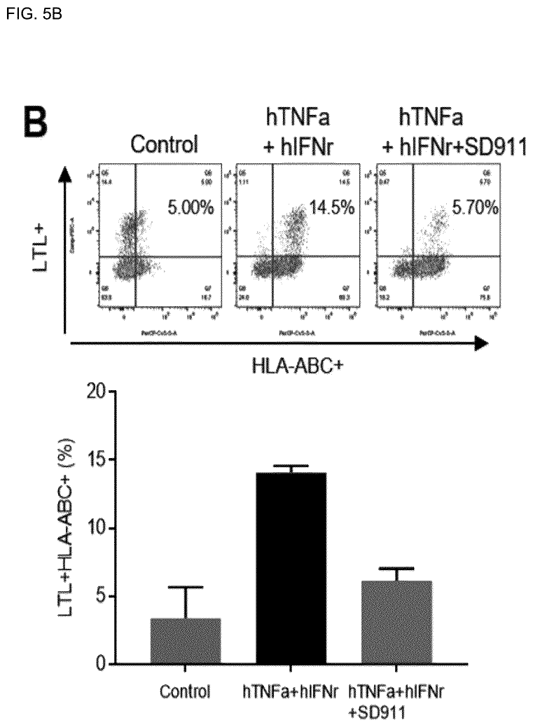

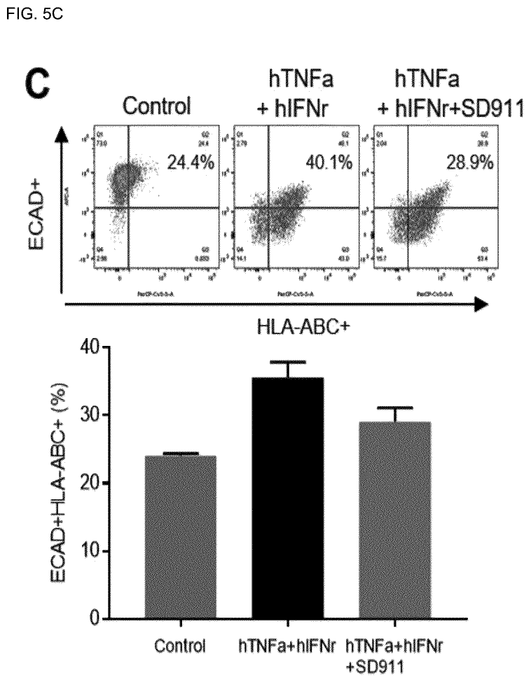

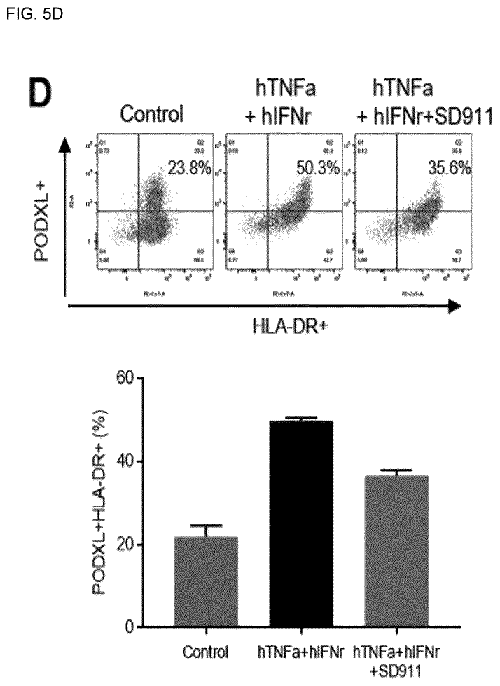

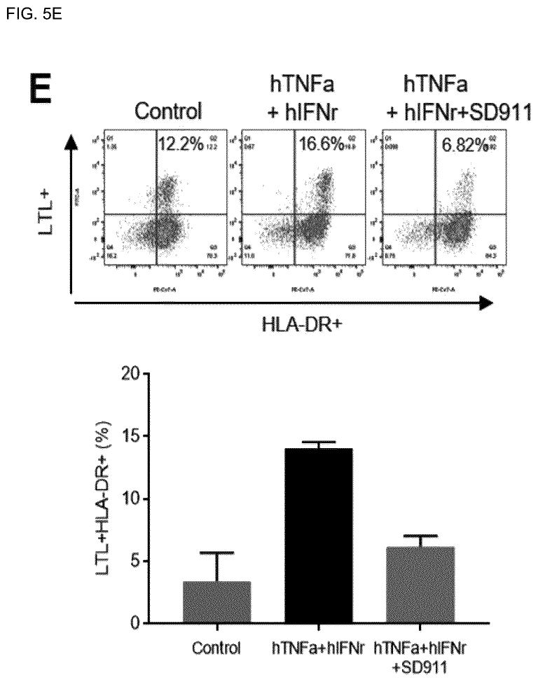

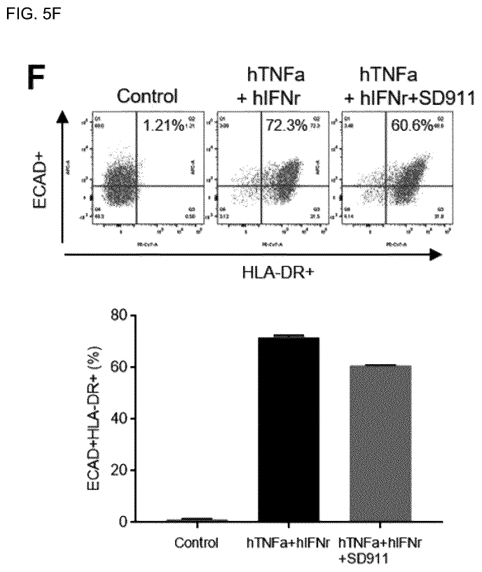

As a result, as illustrated in FIGS. 5A to 5F, it was confirmed that when the cytokines TNF-α and INFγ were treated, the positive cell ratio of the nephron marker increased, and when SD911 was treated at a concentration of 50 μg/mL, the positive cell ratio of the nephron marker significantly decreased.

### <Example 4> Confirmation of SD911 Effect in Kidney Transplantation Rejection Model

### <4-1> Production of Kidney Transplantation Animal Model

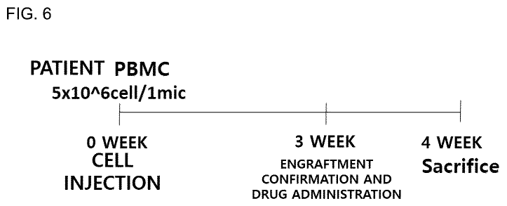

In order to produce a kidney transplantation rejection model, peripheral blood mononuclear cells (PBMCs) derived from transplantation rejection patients (kidney transplantation rejection patients) were intravascularly injected at a cell concentration of 5×10⁶/mice, and then after 3 weeks, engraftment of transplantation rejection patient cells in the blood was confirmed, and a kidney transplantation rejection model was constructed. As a control group, normal human PBMCs were injected (FIG. 6).

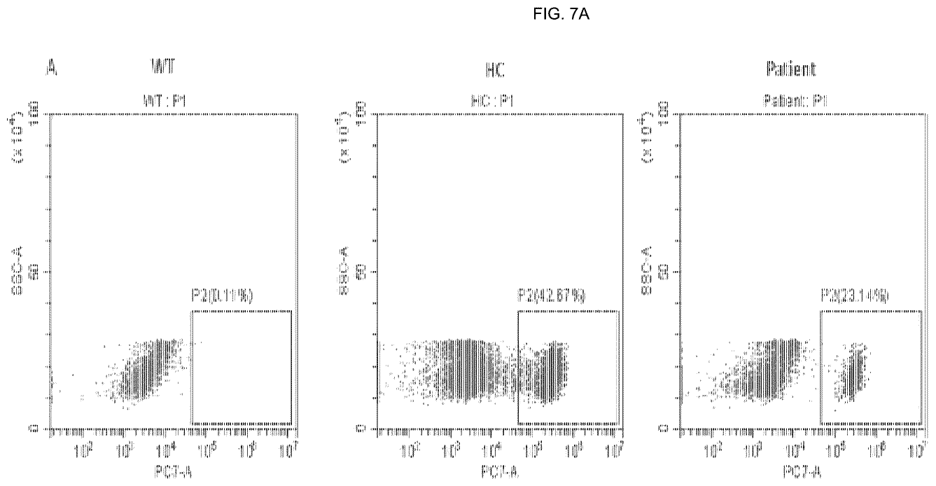

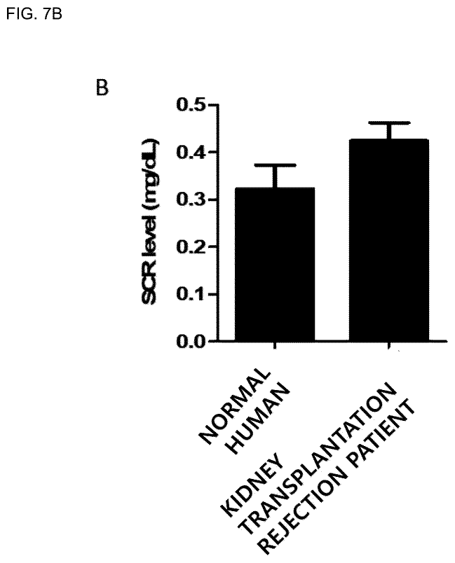

In order to confirm whether the kidney transplantation rejection model was effectively constructed, the engraftment of transplantation rejection patient-derived PBMCs in the mouse model was confirmed by flow cytometry using Human CD4 staining, and a concentration of serum creatinine (SCR) was measured using serum. As a result, it was confirmed that human PBMCs were effectively engrafted into mice, and it was confirmed that the concentration of serum creatinine (SCR) was significantly higher in the patient-derived PBMC injection group than in the normal human-derived PBMC administration group, and thus, it was specifically confirmed that the kidney transplantation rejection model was constructed (FIGS. 7A and 7B).

### <4-2> Confirmation of Suppression of Cell Infiltration and Kidney Damage by SD 911

At 3 weeks after PBMC administration to the kidney transplantation rejection model prepared in Example 4-1, the mice were treated with physiological saline (vehicle) and SD911 (300 μg/mice) and sacrificed at 4 weeks and then the kidneys of the mice were extracted. The kidney was stained using Hematoxylin and eosin (H&E) reagent, and cell infiltration was observed in the glomerulus.

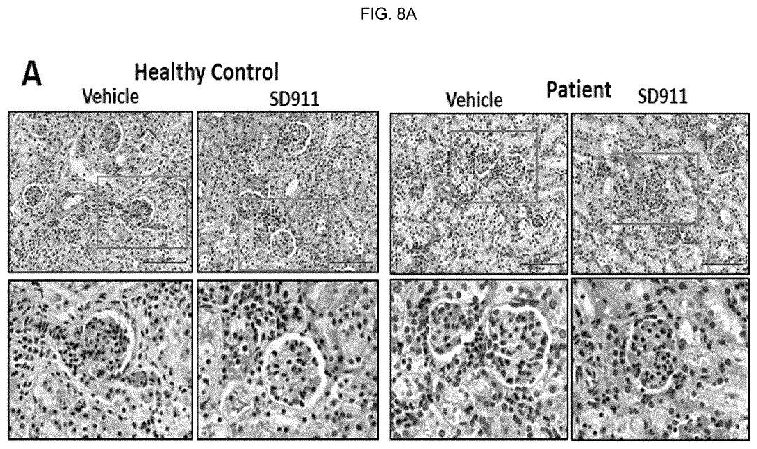

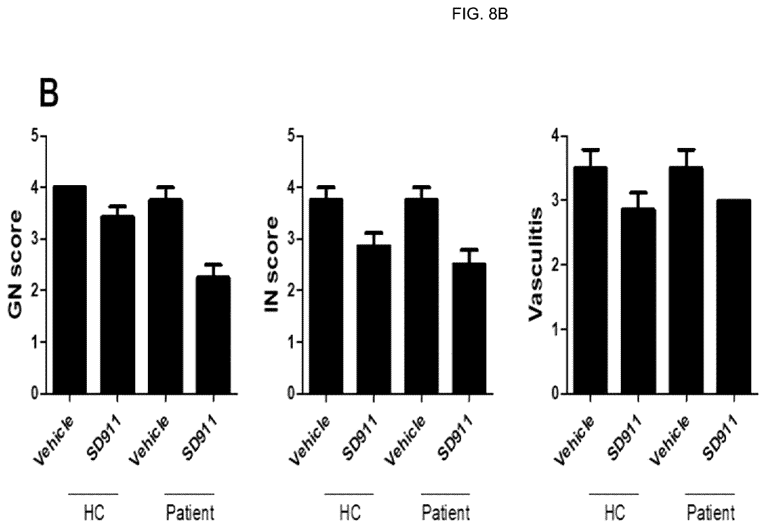

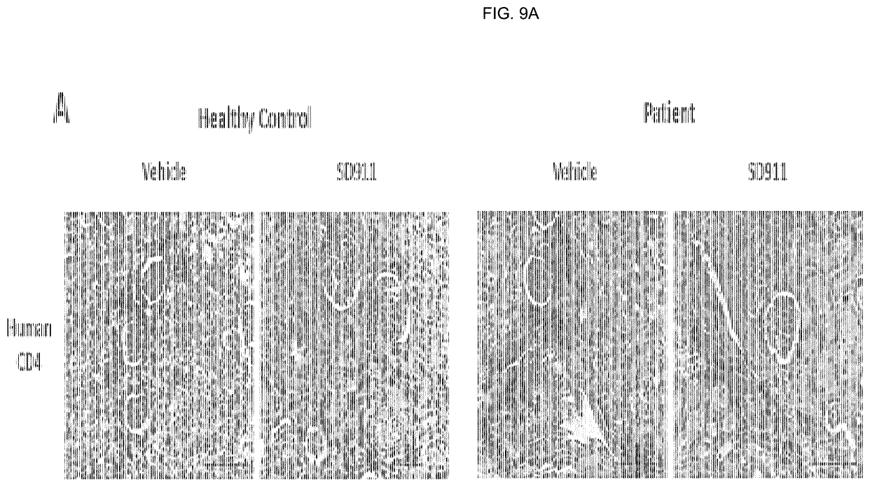

As a result, as illustrated in FIGS. 8A and 8B, it was confirmed that when treated with SD911, as compared with the vehicle group, the human PBMC infiltration was suppressed and the kidney damage was suppressed.

### <4-3> Confirmation of Pathogenic T-cell Infiltration Control in Renal Tissue of SD 911

Immunohistochemical staining was performed to confirm whether CD4+T (CD4+), subtype of human immune cells, has been infiltrated in the renal tissue extracted in Example 4-2. The extracted tissue was fixed with formalin, and then embedded in paraffin to generate slices with a thickness of 5 μm. In order to observe immune cells in the tissue, immunohistochemical analysis was performed by reacting with a human CD4 antibody on the slice slide.

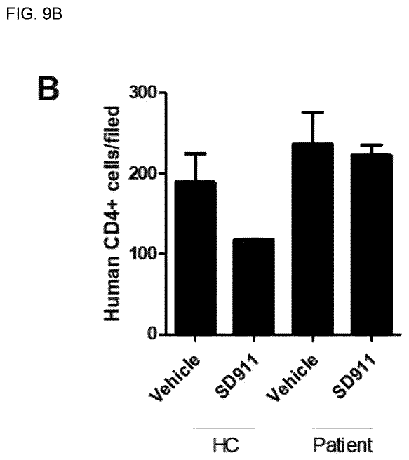

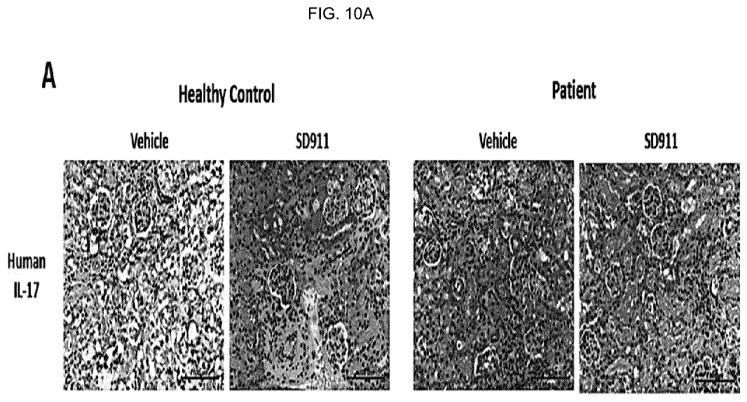

As a result, it was confirmed that when SD911 was treated, the infiltration of CD4-positive cells was significantly reduced in a group injected with PBMCs of patients with kidney transplantation rejection, which was reduced compared to a group injected with normal human PBMCs (FIGS. 9A and 9B).

### <4-4> Confirmation of IL-17 Infiltration Control in Renal Tissue of SD 911

Immunohistochemical staining was performed to confirm whether IL-17, an inflammatory cytokine, has been infiltrated into the renal tissue extracted in Example 4-2. The extracted tissue was fixed with formalin, and then embedded in paraffin to generate slices with a thickness of 5 μm. In order to observe immune cells in the tissue, immunohistochemical analysis was performed by reacting with a human IL-17 antibody on the slice slide.

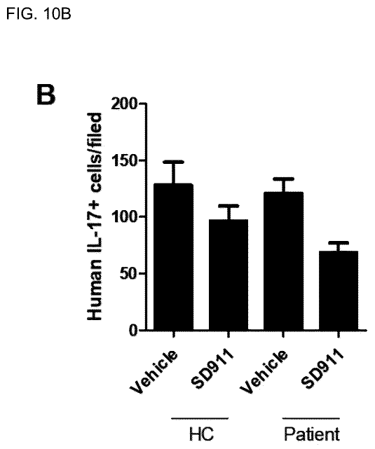

As a result, it was confirmed that when SD911 was treated, the infiltration of IL-17-positive cells was significantly reduced in a group injected with PBMCs of patients with kidney transplantation rejection, which was reduced compared to a group injected with normal human PBMCs (FIGS. 10A and 10B).

Accordingly, the novel compound SD911 of the present invention was identified to suppress the migration of immune cells, increase the engraftment and viability of a grafted tissue, and regulate immune cells. In addition, it was found in a kidney transplantation model that the compound had a transplantation rejection effect by suppressing the engraftment of human PBMC, damage to renal tissues, and the expression of the inflammatory cytokine IL-17.

1. A method for treating transplant rejection comprising administering a pharmaceutical composition comprising a compound represented by Chemical Formula 1 below or a pharmaceutically acceptable salt thereof as an active ingredient to a subject in need thereof.

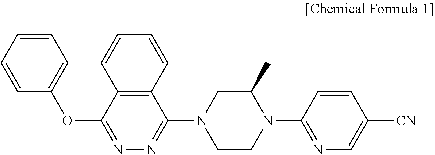 [SMILES: C[C@@H]1CN(c2nnc(Oc3ccccc3)c3ccccc23)CCN1c1ccc(C#N)cn1]

2. The method of claim 1, wherein the transplantation rejection is at least one kind of transplantation rejection selected from the group consisting of cells, blood, tissues and organs.

3. The method of claim 2, wherein the transplantation rejection is at least one kind selected from the group consisting of bone marrow transplant, heart transplant, corneal transplant, intestinal transplant, liver transplant, lung transplant, pancreas transplant, kidney transplant, and skin transplant rejections.

4. The method of claim 1, wherein the transplantation rejection includes graft-versus-host disease (GVHD).

5. The method of claim 1, wherein the composition suppresses the migration of immune cells.

6. The method of claim 1, wherein the composition increases the viability of a graft.

7. The method of claim 1, wherein the composition regulates immune cells.

8. The method of claim 1, wherein the composition inhibits the differentiation or activity of one or more T cells selected from the group consisting of Th1, Th2 and Th17.

9. The method of claim 1, wherein the composition increases the differentiation or activity of Treg.

10. The method of claim 1, wherein the composition inhibits the expression of HLA induced with cytokines.

11. The method of claim 1, wherein the composition inhibits the tissue damage.

12. The method of claim 11, wherein the tissue is at least one kind of tissue selected from the group consisting of bone marrow, heart, cornea, intestine, liver, lung, pancreas, kidney and skin.

13. The method of claim 1, wherein the composition suppresses the infiltration of immune cells.

14. A method for improving immunosuppression after transplantation administering a composition comprising the compound or the pharmaceutically acceptable salt thereof of claim 1 as an active ingredient.

15. (canceled)

16. A method for treating transplantation rejection, comprising administering the compound or the pharmaceutically acceptable salt thereof of claim 1 thereof to a subject.
